(19) United States
(12) Patent Application Publication
(10) Pub. No.: US 2024/0261485 A1
(43) Pub. Date: Aug. 8, 2024

(54) Vascular Access Device

(71) Applicant: UNM Rainforest Innovations, Albuquerque, NM (US)

(72) Inventors: Augustus Pedrotty, Albuquerque, NM (US); Sam Pedrotty, Albuquerque, NM (US)

(73) Assignee: UNM Rainforest Innovations, Albuquerque, NM (US)

(21) Appl. No.: 18/609,962

(22) Filed: Mar. 19, 2024

Related U.S. Application Data
(63) Continuation of application No. 17/378,594, filed on Jul. 16, 2021, now abandoned.
(60) Provisional application No. 63/052,875, filed on Jul. 16, 2020.

(51) Int. Cl. A61M 1/36; A61M 39/02

(52) U.S. Cl. CPC A61M 1/3655; A61M 39/0247; A61M 2039/0258; A61M 2039/0261; A61M 2039/0294

(57) ABSTRACT

A method of using a vascular access device that prevents needle infiltration, provides a repeatable same-site access, and simplifies cannulation all while being minimally invasive and allows patients to self-cannulate.

### RELATED APPLICATIONS

This application is a continuation of U.S. Ser. No. 17/378,594 filed on Jul. 16, 2021, which claims priority to U.S. Provisional Application No. 63/052,875, filed on Jul. 16, 2021, both of which are incorporated herein in their entirety.

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### BACKGROUND OF THE INVENTION

Hemodialysis, the most common form of renal replacement therapy (RRT), requires access to the vascular system. The primary forms of vascular access for hemodialysis are arteriovenous fistulae (AVF), arteriovenous grafts (AVG), and tunneled dialysis catheters (TDC). AVFs have a poor rate of maturation and often require significant revision or repeated interventions to maintain patency. AVGs mature more quickly for use but are prone to thrombosis and require an even greater rate of endovascular interventions to maintain patency than AVFs. Finally, TDCs have an unacceptably high rate of life-threatening bloodstream infections.

The most common vascular access devices or methods used in hemodialysis are AVFs and catheters. Other access approaches and devices exist, including polymer grafts and ports. Common access methods are prone to failure and often require repair or even replacement, leading to a significant patient burden and an annual cost of more than one billion dollars in the United States alone.

The systemic issue for port-type devices is their use of a catheter to connect the port to the vascular system. While ports may address infection relative to standard catheter use, they still suffer the same patency issues.

### BRIEF SUMMARY OF THE INVENTION

In one embodiment, the present invention concerns a novel vascular access device that overcomes many of the shortfalls of current vascular access devices and methods.

In other embodiments, the present invention provides a device that prevents needle infiltration, provides a repeatable same-site access, and simplifies cannulation all while being minimally invasive. These features reduce the damage repeated cannulation does to the target vessel, prolonging its useful life, and minimizing interventions. This device also allows more patients to self-cannulate, giving them more control over their treatment. Finally, the device's flexibility and placement around (but not in) a vessel allow it to be used on existing access (like a fistula) or on a new vessel where it could enable a removable Scribner-type shunt. This easy to use and robust access offers a significant improvement in patient quality of life and enables hemodialysis systems that are not feasible with current access (like wearable dialysis systems).

In other embodiments, the present invention provides a novel vascular access device that overcomes many of the shortfalls of current vascular access devices and methods, significantly improving patient quality of life. The device is made of biocompatible material, features simple construction and installation, is easy to use, can be used with existing AVFs, and can be quickly manufactured to match a patient's vasculature at low cost.

In other embodiments, the present invention provides a vascular access device comprising: a center channel, a needle guide, and tissue anchors.

In other aspects, the present invention provides a vascular access device wherein the tissue anchors are opposingly located side wings that extend perpendicularly away from the center channel.

In other aspects, the present invention provides a vascular access device wherein the opposingly located side wings are adapted to prevent lateral movement and rolling.

In other aspects, the present invention provides a vascular access device wherein the needle guide is funnel-shaped and includes an angled bore that runs through the funnel.

In other aspects, the present invention provides a vascular access device further including a body defined by upper and lower sections that are releasably connectable.

In other aspects, the present invention provides a vascular access device wherein the upper section defines one half of the channel and the lower section defines the other half of the channel and the channel is completely formed when the sections are connected.

In other aspects, the present invention provides a vascular access device wherein the side wings are comprised of a first piece and second piece, the first piece connected to the upper section and the second piece connected to the lower section.

In other aspects, the present invention provides a vascular access device wherein the first piece includes a boss having a fastener opening and the second piece includes internal threads that align with the fastener opening.

In other aspects, the present invention provides a vascular access device wherein the boss of the first piece is rounded, and the second piece is rectangular.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1 illustrates an embodiment of the present invention.

FIG. 2 is a side view of the embodiment shown in FIG. 1.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention

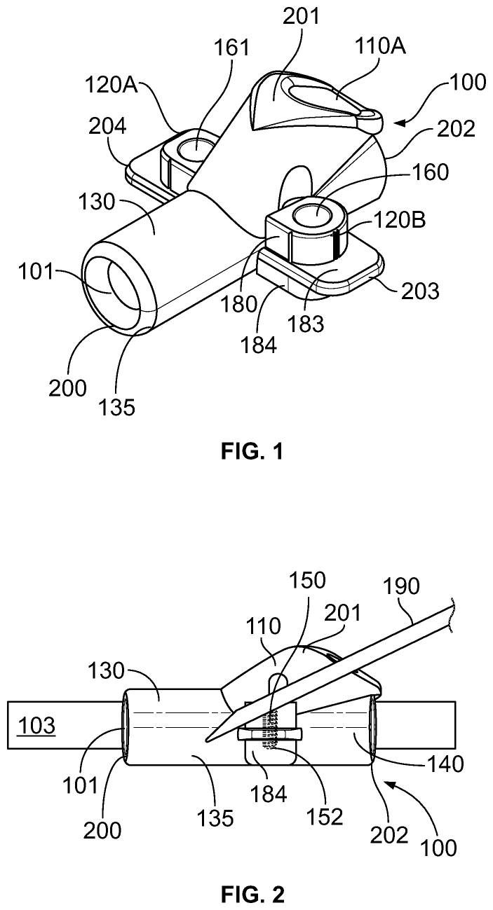

In a preferred embodiment, as shown in FIGS. 1 and 2 the present invention provides a vascular access device 100 that functions as a needle guide while also reinforcing existing vasculature. The device features a center channel 101, that envelopes a blood vessel 103, and a funnel-like needle guide 110.

As shown, the access point of the device is a funnel 110 having an opening therein that provides a secure fit for the desired needle gauge. the funnel makes access easy as it's readily identified through the skin and will correct the orientation of the needle as it enters the vessel. The funnel also provides support to the needle, so the vessel won't be damaged, and the needle won't otherwise dislodge if the needle/access point/blood circuit/etc. is disturbed during treatment.

In a preferred embodiment, the channel forms a complete vessel enclosure by completely enclosing the vessel. This will help prevent painful and potentially life threatening vessel infiltration.

In other embodiments, the present invention may include side wings 120A and 120B that function as tissue anchors. The anchors set the device into surrounding tissue, preventing it from moving laterally or rolling, which would make it unusable (the access point would be at the bottom in that case).

The body of device 100 is split into two pieces or sections along the longitudinal axis 140 of the device (the upper section 130, which includes needle guide 110A, and lower section 135, as shown in FIG. 2, making installation easy. In other aspects, longitudinal axis 140 splits channel 101 in equal halves along its length.

In another embodiment, the halves are releasably connected by one or more fasteners 150 that engage internal threads 152. The fasteners are sized to fit within openings 160 and 161 in the anchors. In a preferred embodiment, socket-head cap screw fasteners of the standard design are used. This reduces cost, accelerates manufacturing, and also improves the ease of implantation while reducing its cost by using standard tools (no specialized tools required).

Tissue anchors 120A and 120B, when in the form of rectangular-shaped side wings, are opposingly located and extend perpendicularly away from center channel 101. In a preferred embodiment, wings 120A and 120B are a two-piece design. A first piece 180, connected to upper half 130, may be in the form having fastener opening 160. A second piece 184, connected to lower section 135, includes internal threads 152 that align with opening 150. Piece 184 and 180 may be rounded and of minimal size to avoid tissue contact and damage.

The device may be constructed entirely from titanium grade 5 6Al-4V (commonly known as medical-grade titanium). As described above, a preferred embodiment of the present invention provides a single-material, two-piece design. This design makes it easier, cheaper, and faster to manufacture than others that use multiple materials or exotic geometries. Also, a smooth, solid surface may be formed on a known biocompatible material, maximizing biocompatibility (unlike other designs that may use techniques with porous surface finishes like 3D printing).

The material is also compatible with all sterilization techniques—no need to use a specialized method (which some designs that incorporate certain plastics have to use).

The two-piece design of the present invention also makes implanting and removal extremely easy, allowing the device to be implanted and, if necessary, removed with minimal impact to the patient. The two halves of the device slide smoothly over and under vessels—no protrusions or other features that could damage vessels and surrounding tissue during implantation. This design specifically makes it easy to check the fit of the device (and of several various sizes) prior to implantation to ensure the best fit and function as it can be easily positioned behind an isolated blood vessel and visually assessed for fit before installation.

The device is installed with a simple, minimally invasive procedure that first involves creating a pocket around the target blood vessel before isolating the vessel. Then, the lower body is slid under the vessel and the upper body is placed on top. After ensuring the device is oriented correctly (with the distal opening of the needle guide pointing in the direction of blood flow in the vessel), two titanium grade 5 6Al-4V M2 screws are used to fasten the device's body halves together. Installation is then complete after the pocket is closed. The device can be accessed immediately, potentially enabling same-day dialysis, unlike other forms of access which requires weeks or months of healing, development, etc. For hemodialysis, two devices may be installed (one for outflow and one for return flow). Both could be placed on a mature AVF. The embodiment may support dialysis from native, virgin vessels (not grafts or AVFs), with one device installed on a vein and the other on the corresponding artery. This would create something akin to a Scribner shunt, albeit with the significant improvement of being able to easily disconnect (and subsequently reconnect) the shunt from the patient's vasculature.

To access the device, the cannulator first prepares the skin to standards typically used for buttonhole cannulation. The ridge at the top of the needle guide is palpated to determine the precise location and orientation. An appropriately-sized standard needle 190 is then driven through the skin just below the ridge at an approximately 30-degree angle from the surface of the skin. As shown in FIGS. 1 and 2, funnel 110 includes a bore 110A that runs through the funnel at a 30-degree angle. The needle is driven until it hits the back of the device. Based on patient comfort and duration of access, the needle can then be further secured with gauze and tape. While the illustration in FIG. 2 shows a 14 gauge needle, the device can be quickly redesigned to accommodate any vessel and needle size. The devices also offer some flexibility where each device size can accommodate a range of vessels and needle sizes. However, the user should still bear in mind the physical limits of the vessel and should not attempt to cannulate with a needle that has a larger diameter than the vessel. To stop access, the needle is removed, and pressure applied to the insertion point on the skin until bleeding stops.

The device is designed in such a way as to minimize the need for maintenance. However, should the need arise, the device's simple construction and installation near the surface of the skin make it easy to get to it and perform any required servicing.

Should the time come for the device to be removed, it has been designed to be removed very easily. After exposing the implanted device, the screws are removed, and the halves of the body are carefully removed from the outside of the vessel. Removal is complete when the incision is closed.

In other embodiments, the vascular access device may be a septum-free design. No septum is used in order to enable the device to be used by any type of needle. Designs with septums must use non-coring needles, which are harder to find than standard needles. Septums can also wear out and introduce foreign material into the bloodstream.

In yet another embodiment, as shown in FIGS. 1 and 2, the device rounded edges has edges 200-204 at locations that may come into contact with a patient. Rounding at certain locations to specific radii that minimize wear on the vessel and surrounding tissue, even as the vessel and surrounding tissue may shift due to the patient's daily activity.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above-described embodiments, methods, and examples, but by all embodiments and methods within the scope and spirit of the disclosure.

What is claimed is:

1. A method of using a vascular access device for hemodialysis comprising the steps of:
a vascular access device, said vascular access device comprised of a body defined by upper and lower sections that are releasably connectable;
said upper section includes a partial center channel and said lower section includes a partial center channel, when said upper and lower sections of said body are connected, a complete center channel is formed;
said upper section includes a two opposing located partial side wings, located perpendicular to said center channel and said lower section includes a partial two opposing located partial side wings located perpendicular to said center channel, when said upper and lower sections of said body are connected, tissue anchors are formed;
said tissue anchors are extend perpendicularly away from said center channel;
said opposingly tissue anchors are adapted to prevent lateral movement and rolling of said body;
said upper section including a needle guide in communication with said center channel;
said needle guide includes an angled bore in communication with said center channel;
said bore includes a funnel-shaped section that transitions into a cylindrical section having a length that matches a needle size and insertion angle to restrain a needle to promote installation, removal and dislodgement.

2. The vascular access device of claim 1 wherein said upper section defines one half of said channel and said lower section defines the other half of said channel and said channel is completely formed when said sections are connected.

3. The vascular access device of claim 1 wherein said side wings are comprised of a first piece and second piece, said first piece connected to said upper section and said second piece connected to said lower section.

4. The vascular access device of claim 3 wherein said first piece includes a boss having a fastener opening and said second piece includes internal threads that align with said fastener opening.
